(19) United States
(12) Patent Application Publication
(10) Pub. No.: US 2003/0078187 A1
(43) Pub. Date: Apr. 24, 2003

(54) TARGETS OF THE MAP KINASE PATHWAY IN THE DEVELOPMENTAL SWITCH IN YEAST

(76) Inventor: HITEN D. MADHANI, SAN FRANCISCO, CA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 09/439,969

(22) Filed: Nov. 12, 1999

Related U.S. Application Data
(60) Provisional application No. 60/108,399, filed on Nov. 13, 1998.
(60) Provisional application No. 60/114,849, filed on Jan. 6, 1999.

(51) Int. Cl. A01N 43/04; A01N 61/00; C12Q 1/68

(52) U.S. Cl. 514/1; 514/23; 435/6

(57) ABSTRACT

Assessment of targets of the MAP kinase pathway in the developmental switch between haploid invasive growth and diploid pseudohyphal development in fungi, and identification of genes that show strong regulation by a MAPK pathway-specific transcription factor, Tec1, are described. Also described are methods of identifying an agent which inhibits the filamentation MAPK pathway in a fungus, and methods of inhibiting filamentation of a fungus or invasion of a host by a fungus.

### RELATED APPLICATIONS

This application claims the benefit of U.S. Provisional Application 60/108,399, filed Nov. 13, 1998, and U.S. Provisional Application 60/114,849, filed Jan. 6, 1999. The entire teachings of these applications are incorporated herein by reference.

### GOVERNMENT SUPPORT

Work described herein was supported, in whole or in part, by National Institutes of Health Grant Number GM 40266. The United States Government has certain rights in the invention.

### BACKGROUND OF THE INVENTION

Two related developmental events, haploid invasive growth and diploid pseudohyphal development, are controlled by the Kss1 MAP kinase pathway in yeast. Haploid invasive growth occurs on rich medium, whereas filamentation in the diploid cell type requires nitrogen starvation. The diploid pathway results in dramatic cell elongation, which is not seen in haploids. These pathways serve as models for similar transitions in pathogenic fungi.

### SUMMARY OF THE INVENTION

Wild yeast are often found in association with plants, particularly rotting fruit. Not surprisingly, many bacterial and plant fungal pathogens secrete pectin-degrading enzymes, including polygalacturonases. These are thought to be key virulence factors. In the bacterial pathogen Erwinia chrysanthemi there exists an elaborate interaction between the host and the pathogen in which the breakdown product of pectin, galacturonic acid, signals large changes in the expression of the pectinolytic machinery. To examine whether a similar interaction with the host might be occurring in yeast, global profiling experiments of gene expression in the presence of polygalacturonic acid or galacturonic acid were carried out.

Described herein is assessment of targets of the MAP kinase (MAPK) pathway in the developmental switch between haploid invasive growth and diploid pseudohyphal development in yeast, and identification of genes that show strong regulation by a MAPK pathway-specific transcription factor, Tec1. Also described herein are results of examination of expression profiles after administration of polygalacturonic acid (the main component of pectin) or galacturonic acid (the breakdown product of pectin), as well as the results of detailed studies of PGUI, a pectinase, which was shown to be the most strongly regulated target of the MAPK pathway.

Also described are global profiling experiments of gene expression in yeast which were carried out to examine whether a host-yeast interaction occurs in which the breakdown product of pectin signals or causes changes in the expression of components of the pectinolytic machinery. As discussed herein, results of these profiling experiments showed that both polygalacturonic acid and galacturonic acid altered gene expression in yeast, and that the patterns were distinct from those that would have been expected from the effects of all other sugars that have been studied in yeast (e.g., glucose, galactose, maltose, etc.), demonstrating the specificity of the response. Galacturonic acid, the breakdown product of pectin, was shown to cause strong repression of TOT10/YEL033W, a gene which is turned on in the filamentation MAPK pathway and is required for invasion and filamentation. Thus, a regulatory circuit in yeast, in which a signal from the host (in the form of or mediated by galacturonic acid) feeds back on the filamentation/invasion pathway, has been identified, and a specific interaction between yeast and its host (e.g., a plant host) has been demonstrated for the first time.

As a result of the work described herein, targets of the MAPK pathway in fungi (e.g., yeast) and, particularly, genes that show strong regulation by Ted1, a MAPK pathway-specific transcription factor, have been identified. These genes and their interaction with or regulation by Tec1 can be targeted in a method of modulating (inhibiting or enhancing) the developmental switch between haploid invasive growth and diploid filamentation. Compounds or molecules which modulate these genes, directly or through their regulation by Tec1, can be identified by means, for example, of an assay in which one or more of the genes (e.g., a gene encoding PGUI) is expressed in an appropriate host cell and the effects of a candidate modulator (inhibitor or enhancer) on its expression are determined. Candidate modulators shown to decrease expression are inhibitors of a gene shown, as described herein, to be regulated by Tec1; candidate modulators shown to increase expression are enhancers of such a Tec1-regulated gene. In addition, the TOT10/YEL033W gene, shown, as described herein, to participate in a regulatory circuit between yeast and a host (e.g., a plant host) can be targeted to modulate (decrease or increase) yeast-host interaction. It can be targeted, for example, to inhibit yeast invasion and/or filamentation and, thus, to inhibit adverse effects of fungi, including pathogenic and nonpathogenic yeast. Inhibitors (or enhancers) of TOT10/YEL033W can be identified, for example, in an assay in which the gene is expressed in an appropriate host cell and the effects of candidate inhibitors (or enhancers) are assessed. Inhibition of TOT10YEL033W, directly or indirectly (e.g., by inhibiting a gene or the product of a gene with which TOT10/YEL033W interacts) will result in inhibition of invasion and/or filamentation. Inhibitors and enhancers of genes regulated by Tec1 and inhibitors of TOT10/YEL033W are the subject of this invention.

Compounds or molecules which activate or inhibit PGUI can also be identified. For example, activators of this pectinase can be identified by expressing PGUI in an appropriate host cell (e.g., a bacterial or yeast cell), contacting the cells with (e.g., by culturing them in the presence of) candidate activators (compounds or molecules to be assessed for their effects on PGUI activity) and determining their effect on PGUI (e.g., whether they enhance or activate PGUI expression or activity, repress or decrease PGUI expression or activity or have no effect). Compounds which enhance or activate PHUI expression or activity are activators; those which repress or decrease its expression or activity are inhibitors). Activators and inhibitors of PGUI are also the subject of this invention.

Also the subject of this invention is a method of inhibiting (totally or partially) invasion of a host, particularly a plant host by a fungus (i.e., a method of inhibiting fungal invasion of a host). In the method, a compound or molecule which inhibits the MAPK pathway or specifically inhibits TOTI0/YELO33W is applied to a host (e.g., by application to a plant surface) in such a manner that it contacts the fungus (e.g., the yeast) and inhibits one or more components of the MAPK pathway, such as TOT10/YELO33W. For example, an inhibitor can be a compound which binds and inhibits TOT10/YELO33W; galacturonic acid; or a mimic of galacturonic acid which represses TOT10/YELO33W. In a specific embodiment, the method of inhibiting fungal invasion of a host comprises contacting a fungus (e.g., a yeast) with a compound which inhibits the MAPK pathway and/or inhibits TOT10/YELO33W, in sufficient quantity that inhibition of the MAPK pathway and/or inhibition of TOT10/YELO33W occurs, thereby inhibiting fungal invasion of the host. In a further embodiment, the host is a plant and the compound is applied to a plant surface (e.g., root, leaf, stem) or seed in such a manner that it contacts the fungus and inhibits (totally or partially) the ability of the fungus to invade.

### BRIEF DESCRIPTION OF THE DRAWINGS

FIG. 1 shows genetic expression profiles of 18 genes regulated by the filamentation MAPK pathway.

FIG. 2 lists MAPK pathway targets.

FIG. 3 summarizes results of systematic knockout experiments.

FIG. 4 is a photograph of results of an assay showing that filamentation MAPK pathway controls pectinolysis via PGUI.

FIG. 5 shows genes selectively induced by the plant-specific carbohydrate polygalacturonic acid and its hydrolysis product.

FIG. 6 shows genes selectively repressed by the plant-specific carbohydrate polygalacturonic acid and its hydrolysis product.

FIG. 7 is a compilation of MAPK data, sorted as TEC1-high copy/tec1Δ.

FIG. 8 shows results of profiling experiments with polygalacturonic acid (PGA) and galacturonic acid (GA), sorted by PGA/YPD.

FIG. 9 shows results of profiling experiments with polygalacturonic acid (PGA) and galacturonic acid (GA), sorted by GA/YPD.

FIG. 10 shows a flow chart of homologous genes induced by the filamentation and mating MAPK pathways.

FIG. 11 shows a listing of genes whose expression is reduced in STE12⁻, STE7⁻ but show greater than double an effect with Tec1.

### DETAILED DESCRIPTION OF THE INVENTION

Described herein is work carried out to identify and study the targets of the MAP kinase pathway in order to understand how signaling cascades control a developmental switch in this Saccharomyces cerevisiae model system. The pathway consists of four kinases Ste20 (PAK), Ste11 (MEKK), Ste7 (MEK) and Kss1 (MAPK), which display both positive and negative control over the pathway, as well as a heterodimeric transcription factor Tec1-STE12. STE7, STE11 and STE20 also participate in the yeast mating MAPK pathway. Global expression patterns in haploid cells under rich medium conditions were examined in the following mutants: wild type tec1Δ Ste12Δ, Ste7Δ, TEC1-overexpression, and STE11-4 (an activated mutant of the MEKK). Expression profiling was carried out using nucleic acid arrays (chips) such as described in WO95/11995. One chip set was used per sample (chips with obvious defects were redone).

18 genes were identified that show strong regulation by the pathway-specific transcription factor Tec1 (i.e. 3.5-20× difference in expression comparing TEC1-overexpression to tec1Δ). Almost all of these also show a consistent dependency on STE7, STE7, and STE12. One gene that was known previously to be regulated by the pathway, FLO11 (which encodes a cell surface protein required for pseudohyphal growth) is the second-most strongly regulated target. Detailed studies were performed on one of these targets, PGU1, which encodes a secreted carbohydrate-destroying enzyme. This enzyme breaks down a key component of plant cell walls, polygalacturonic acid (which is the main component of pectin).

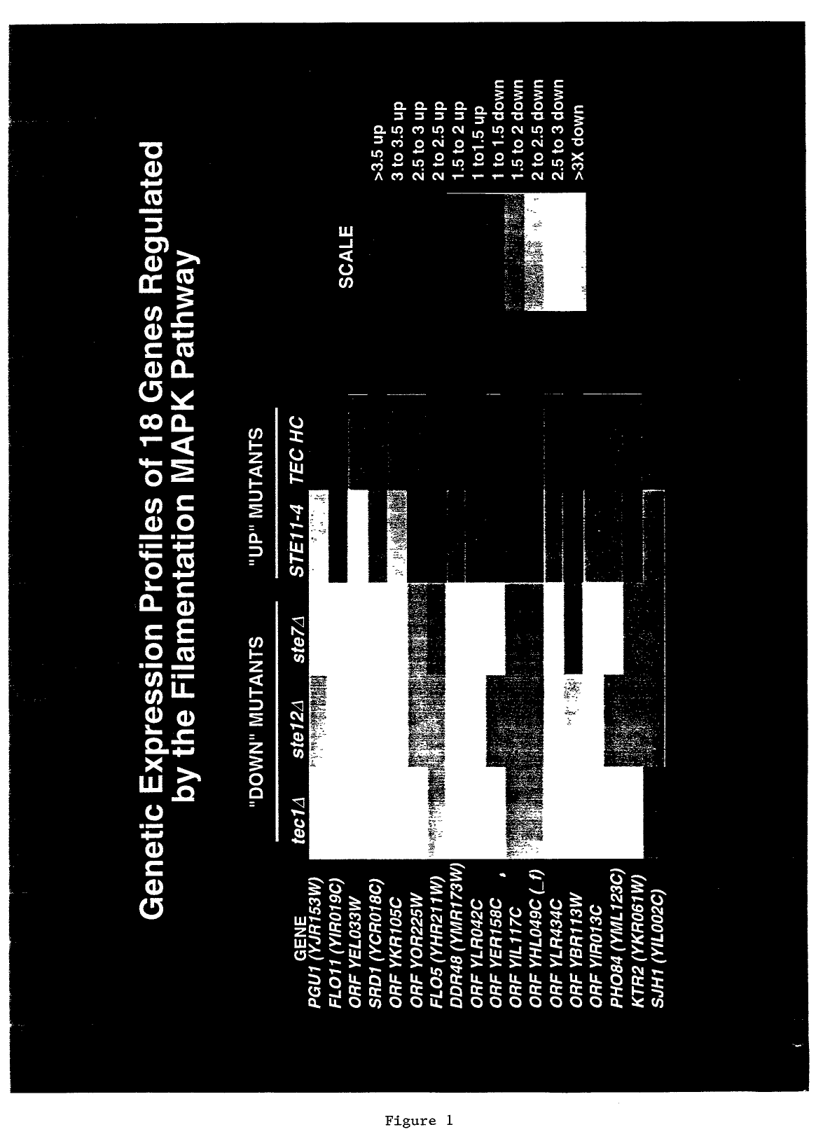

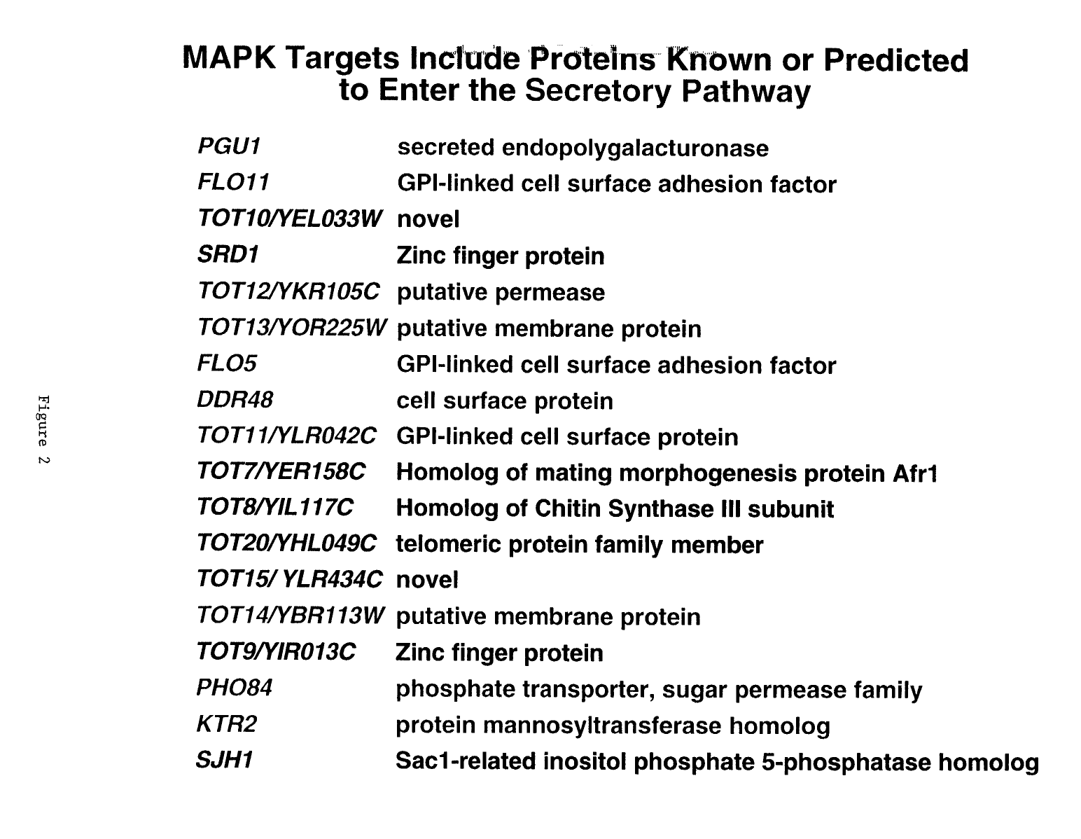

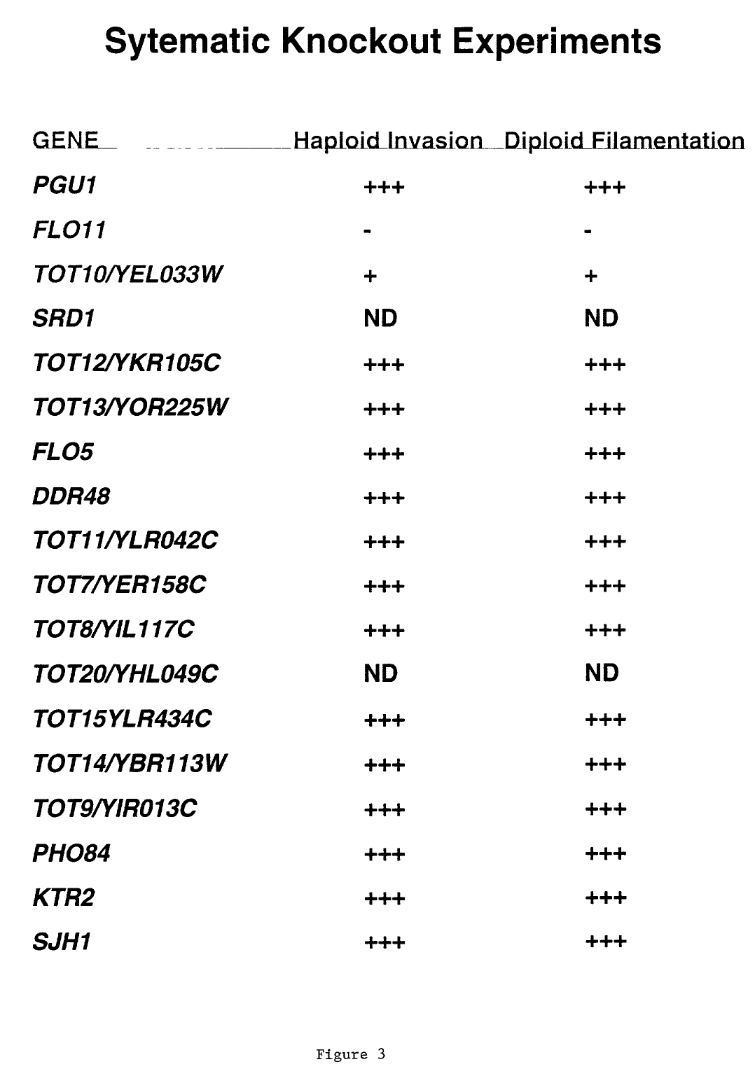

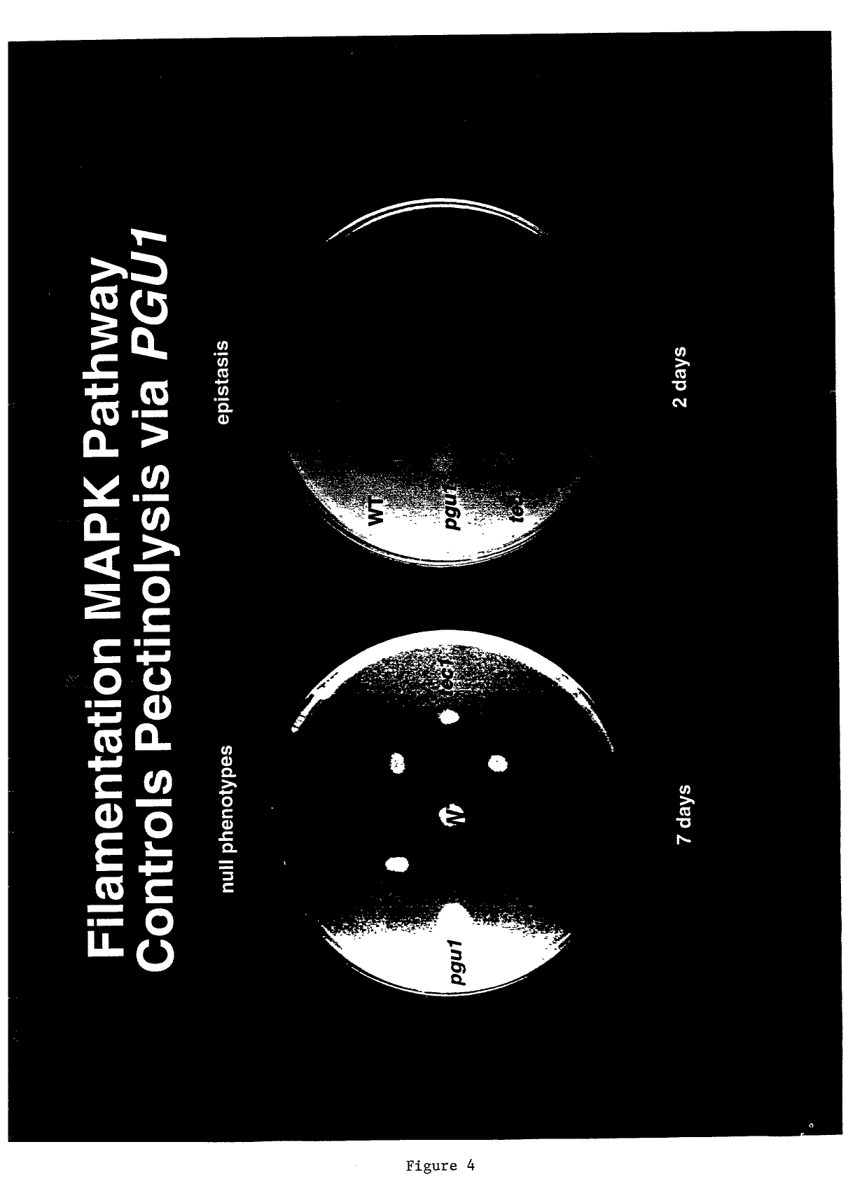

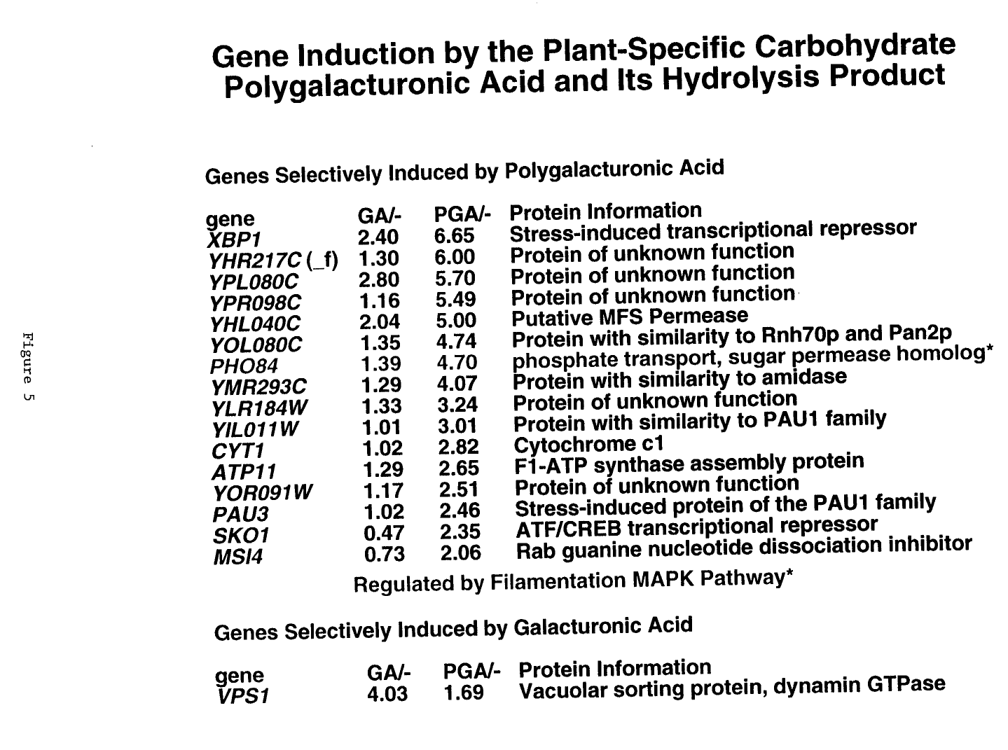

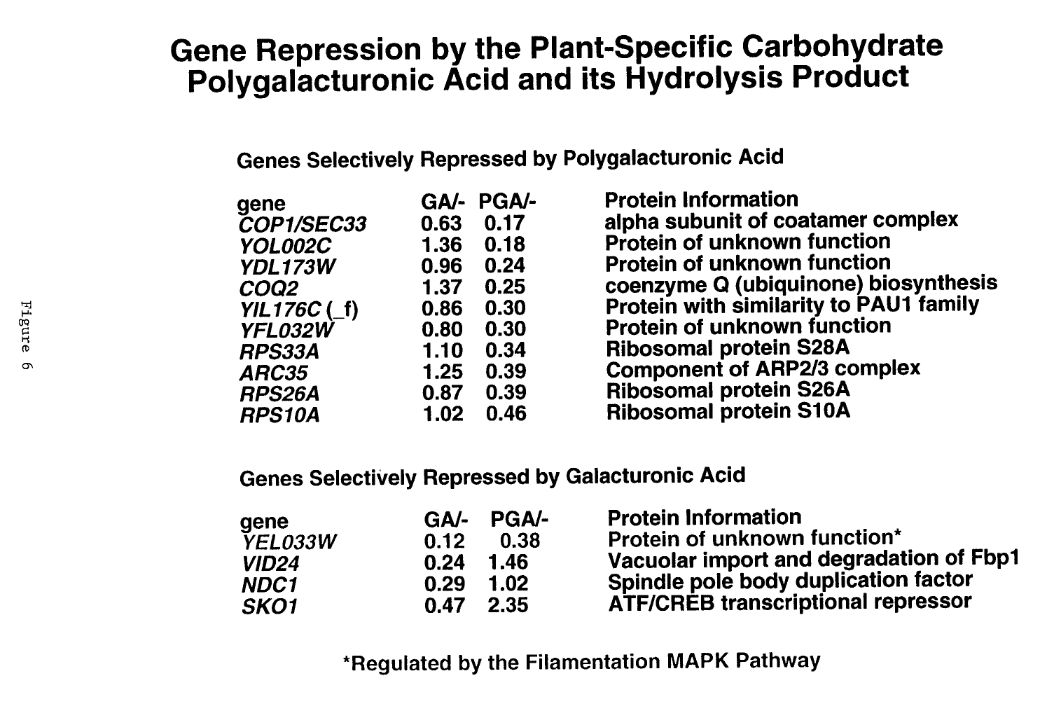

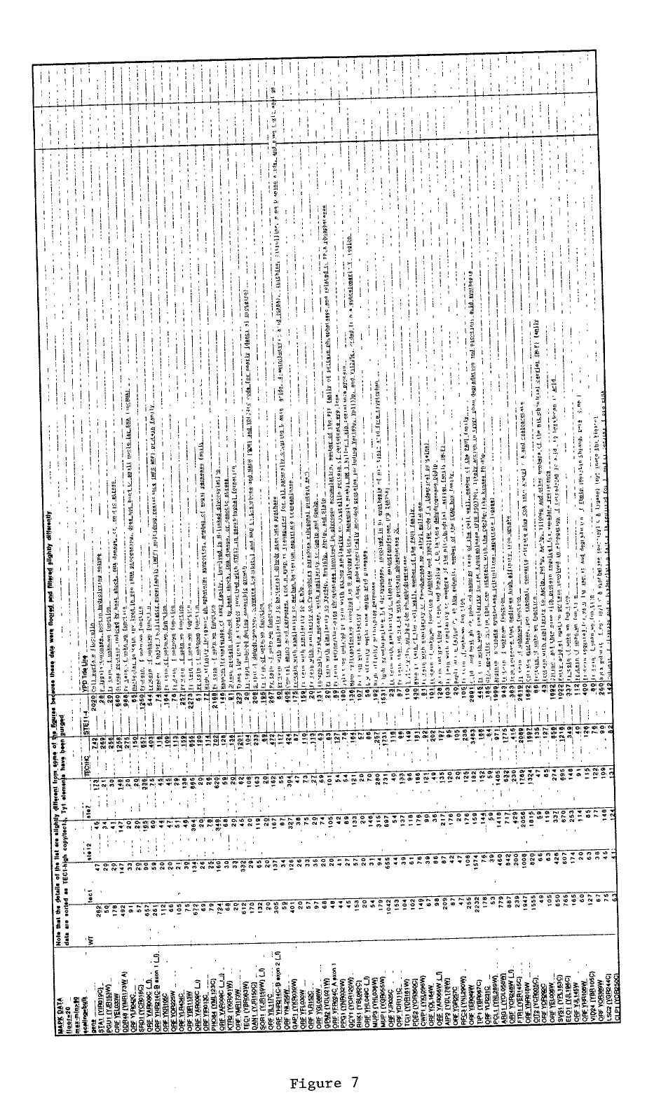

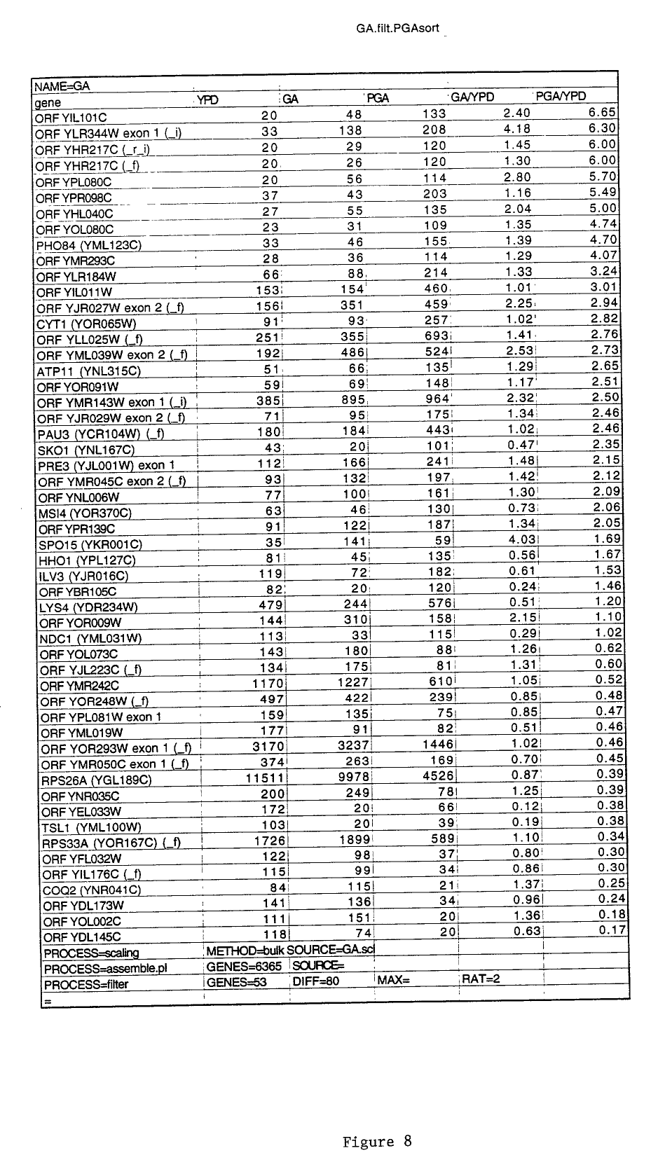

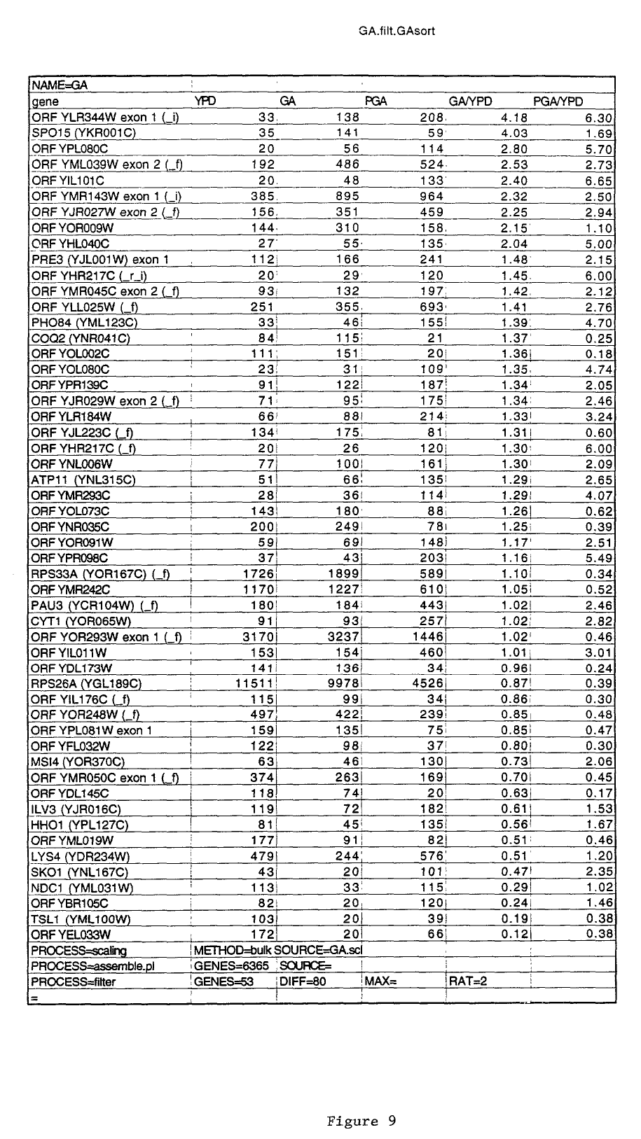

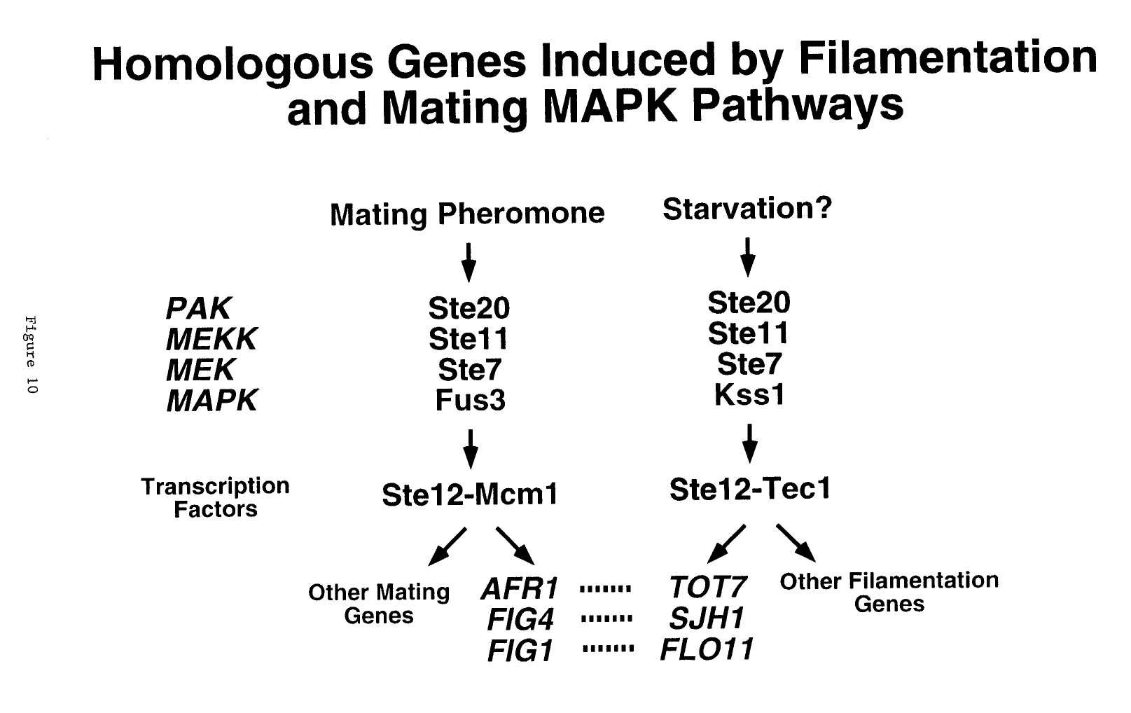

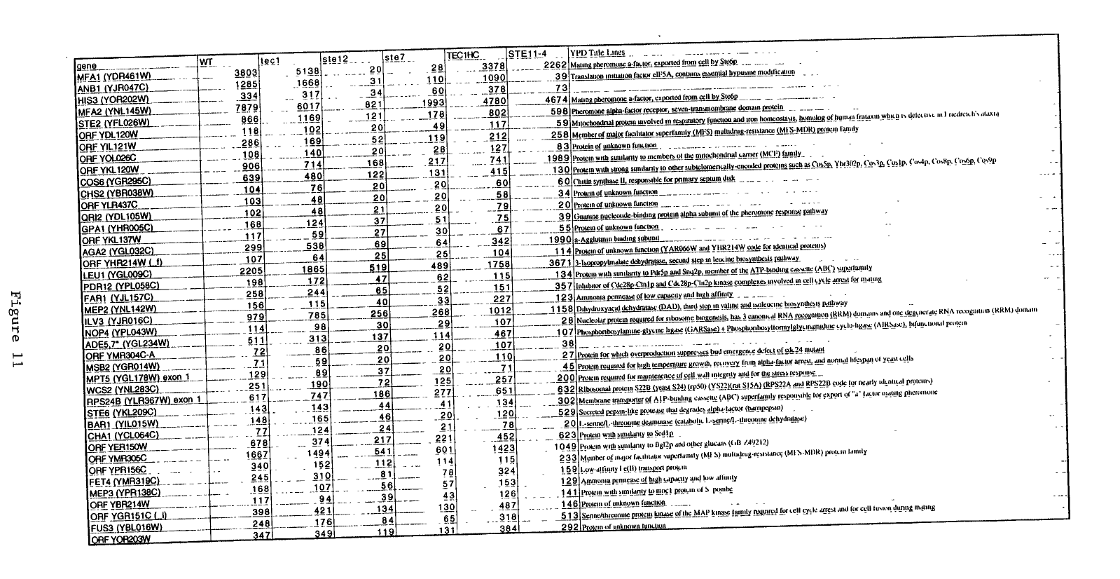

Remarkably, galacturonic acid, the breakdown product of pectin, causes the strong repression of a gene, TOT10/YEL033W, which is turned on in the filamentation MAPK pathway and which these results have shown is required for invasion and filamentation. Thus, work described herein has identified a new regulatory circuit in yeast in which a signal from the host feeds back on the filamentation/invasion pathway. This is the first demonstration of a specific interaction between yeast and its plant host. FIGS. 1-11 show the data in detail.

Work described herein provides an analysis of data from haploid strains grown in rich medium conditions, and in diploid cells under nitrogen starvation conditions; that is, the conditions that promote pseudohyphal cells. Portion os this work was carried out to assess whether pseudohyphal cells respond to MAPK signaling differently compared to haploid cells. The experiments described compare the expression of strains overexpressing the transcription factor Tec1 to those lacking it. They extend the assessment of targets of the MAP kinase pathway in a yeast developmental switch in haploid cells to examination of signaling in diploid cells. The data (Tables 1 and 2) were analyzed using a floor of 20 and a maximum-minimum filter of 80. Genes showing a greater than two-fold change in duplicate samples are listed. The results indicate that a largely different set of genes is induced by the MAPK pathway during pseudohyphal conditions. One striking exception is the FLO11 gene, which is the gene most strongly induced both in haploids and diploids by the pathway. The other genes fall mainly into the categories of cell-cycle regulated genes (such as histones and PCNA), nitrogen scavenging factors (e.g., Dur3, Car2). A number of other genes are regulated that do not at present fit into any pattern.

Accordingly, the invention relates to a method of inhibiting invasion of a host by a fungus, comprising contacting the fungus with a compound which inhibits expression of a gene expressed in the filamentation MAPK pathway and which enhances the filamentation MAPK pathway, in sufficient quantity that inhibition of the expression of the gene occurs, thereby inhibiting invasion of the host by the fungus. In one embodiment, the host is a plant, and the compound is applied to a plant surface (e.g., a leaf, a root, a stem, a flower) in such a manner that it contacts the fungus. An effective amount of the compound can be determined empirically by assessing expression levels of the gene to be inhibited. In a preferred embodiment, the gene is TOT10/YELO33W. In one embodiment, the fungus is a yeast, such as Saccharomyces cerevisiae.

Agents for use in the methods of the invention include nucleic acid molecules (e.g., antisense), polypeptides and proteins, antibodies and small organic molecules. Suitable formulations of agents for use in this invention can include, for example, powders, liquids, aerosols, gels and other formulations known to the skilled artisan. The present invention also pertains to pharmaceutical compositions comprising agents identified according to the invention for use in the treatment of fungal invasion. For instance, the agent identified according to the present invention can be formulated with a physiologically acceptable medium to prepare a pharmaceutical composition. The particular physiological medium may include, but is not limited to, water, buffered saline, polyols (e.g., glycerol, propylene glycol, liquid polyethylene glycol) and dextrose solutions. The optimum concentration of the active ingredient(s) in the chosen medium can be determined empirically, according to procedures well known to medicinal chemists, and will depend on the ultimate pharmaceutical formulation desired. In organisms other than plants, methods of administration of pharmaceutical compositions include, but are not limited to, intradermal, intramuscular, intraperitoneal, intravenous, subcutaneous, oral and intranasal. Other suitable methods of introduction can also include rechargeable or biodegradable devices and slow release polymeric devices. The pharmaceutical compositions of this invention can also be administered as part of a combinatorial therapy with other agents.

The invention also relates to a method of inhibiting invasion of a host by a fungus, comprising contacting the fungus with a compound which alters activity of a gene product encoded by a gene expressed in the filamentation MAPK pathway, in sufficient quantity that alteration of the activity of said gene product occurs, thereby inhibiting invasion of the host by the fungus. For example, if the gene is one whose expression enhances (e.g., increases or potentiates) the filamentation MAPK pathway (e.g., a positive regulator of the pathway), then the compound should inhibit the expression of that gene. As used herein, inhibition is intended to included both qualitative and quantitative reduction, including complete abolishment. Conversely, if the gene is one whose expression inhibits (e.g., decreases or interferes with) the filamentation MAPK pathway (e.g., a negative regulator of the pathway), then the compound should enhance the expression of that gene. As used herein, enhancement is intended to include any qualitative or quantitative increase. For example, the gene can be TOT10YELO33W.

Expression vectors for use in the invention typically contain a nucleic acid sequence of a gene of interest operably linked to at least one regulatory sequence. “Operably linked” is intended to mean that the nucleotide sequence is linked to a regulatory sequence in a manner which allow expression of the nucleic acid sequence. Regulatory sequences are art-recognized and can be selected according to the host cell and type of expression (e.g., constitutive) to be obtained. Accordingly, the term “regulatory sequence” includes promoters, enhancers, and other expression control elements which are described in Goeddel, Gene Expression Technology: Methods in Enzymology 185, Academic Press, San Diego, Calif. (1990). It should be understood that the design of the expression vector may depend on such factors as the choice of the host cell to be tranformed and/or the type of protein desired to be expressed.

Prokaryotic and eukaryotic host cells transfected by the described vectors are also provided by this invention. For instance, cells which can be transfected with the vectors of the present invention include, but are not limited to, bacterial cells such as E. Coli, insect cells (baculovirus), or mammalian cells such as Chinese hamster ovary cells (CHO). Ligating the polynucleotide sequence into a gene construct, such as an expression vector, and transforming or transfecting into hosts, either eukaryotic (avian, insect or mammalian) or prokaryotic (bacterial cells), are standard procedures (see, for example, Broach, et al., Experimental Manipulation of Gene Expression, ed. M. Inouye (Academic Press, 1983) p. 83; Molecular Cloning: A Laboratory Manual, 2nd Ed., ed. Sambrook et al. (Cold Spring Harbor Laboratory Press, 1989) Chapters 16 and 17).

The invention also relates to a method of identifying an agent which inhibits the filamentation MAPK pathway in a fungus, comprising the steps of providing an expression vector comprising a nucleic acid molecule of a gene which is expressed in the filamentation MAPK pathway; transforming a suitable host cell with said expression vector under conditions suitable for expression of said gene contacting said host cell with an agent to be tested; and comparing the expression of said gene in the presence of the agent with the expression of said gene in the absence of said agent, wherein if the expression of said gene is lower in the presence of the agent than in the absence of the agent, then the agent is an inhibitor of the filamentation MAPK pathway in a fungus. In one embodiment, the gene is TOT10/YELO33W.

Genes which are expressed in the filamentation MAPK pathway can be identified by standard methods in the art. In one embodiment, the gene is identified by expression profiling as having repressed expression in the presence of galacturonic acid as compared with in the absence of galacturonic acid. In another embodiment, the gene can be identified by expression profiling as being expressed in haploid fungal cells and not expressed in diploid fungal cells, or as being repressed by Tec1 expression.

The invention also relates to a method of inhibiting fungal filamentation, comprising contacting the fungus with a compound which inhibits expression of a gene expressed in the filamentation MAPK pathway, in sufficient quantity that inhibition of the expression of the gene occurs, thereby inhibiting filamentation by the fungus.

The invention further relates to a method of identifying an agent which modulates PGUI gene expression, comprising the steps of providing an expression vector comprising a nucleic acid molecule encoding PGUI; transforming a suitable host cell with said expression vector under conditions suitable for expression of PGUI; contacting said host cell with an agent to be tested; and comparing the expression of PGUI in the presence of the agent with the expression of PGUI in the absence of said agent, wherein a difference in the expression of PGUI in the presence of the agent as compared with in the absence of the agent indicates that the agent modulates PGUI expression.

The invention also includes a method of reducing the adverse effects of fungal invasion of a host, comprising administering to the host an effective amount of an agent which inhibits PGUI expression in the fungus.

The invention further includes a method of inhibiting invasion of a host by a fungus, comprising contacting the fungus with a compound which enhances expression of a gene expressed in the filamentation MAPK pathway and which inhibits the pathway, in sufficient quantity that enhancement of the expression of the gene occurs, thereby inhibiting invasion of the host by the fungus.

While this invention has been particularly shown and described with references to preferred embodiments thereof, it will be understood by those skilled in the art that various changes in form and details may be made therein without departing from the spirit and scope of the invention as defined by the appended claims.

TABLE 1
PSEUDOHYPHAL CONDITIONS

| gene | Information |
|---|---|
| FLO11 (YIR019C) | GPI-anchored cell surface flocculin req'd for |
| | invasion |
| DUR3 (YHL016C) | Urea Permease |
| HTA2 (YBL003C) | Histone H2A |
| HTB2 (YBL002W) | Histone H2B |
| ORF YNL300W | GPI-anchored S/T rich protein |
| ORF YOL162W | Allantoate permease family |
| OFF YLL057C | Similar to E. coli taurine dioxygenase |
| SVS1 (YPL163C) | S/T rich protein req'd for vanadate resistance |
| ORF YOL163W | Allantoate permease familhy |
| CAR2 (YLR438W) | Ornithine aminotransferase, arginine catabolism |
| TSL1 (YML100W) | Trehalose-6-phosohate synthase/phosphatase subunit |
| PRY2 (YKR013W) | Homolog of Plant Pathogen-Induced Gene |
| POL30 (YBR088C) | PCNA, DNA Replication, Repair and Cell Cycle |
| | Factor |
| PDC6 (YGR087C) | Pyruvate decarboxylase: isobutyl alcohol formation |
| ORF YOR247W | S/T rich protein related to Svs1 |

Floor = 20, max − min > 80, max/min > 2, TEC1HC/tec1Δ > 2 for both chip sets

TABLE 2
PSEUDOHYPHAL CONDITIONS

| gene | tec1KO A | TEC1HC A | tec1KO B | tec1HC B |
|---|---|---|---|---|
| FLO11 (YIR019C) | 46 | 505 | 61 | 471 |
| DUR3 (YHL016C) | 20 | 105 | 53 | 116 |
| HTA2 (YBL003C) | 29 | 145 | 32 | 180 |
| HTB2 (YBL002W) | 122 | 559 | 184 | 617 |
| ORF YNL300W | 349 | 1281 | 340 | 1031 |
| ORF YOL162W | 44 | 160 | 66 | 151 |
| ORF YLL057C | 64 | 227 | 63 | 243 |
| SVS1 (YPL163C) 129 | 453 | 112 | 370 | |
| ORF YOL163W | 53 | 160 | 43 | 179 |
| CAR2 (YLR438W) | 49 | 138 | 43 | 165 |
| TSL1 (YML100W) | 148 | 399 | 175 | 373 |
| PRY2 (YKR013W) | 436 | 1148 | 472 | 1012 |
| POL30 (YBR088C) | 129 | 313 | 103 | 261 |
| PDC6 (YGR087C) | 129 | 276 | 78 | 270 |
| ORF YOR247W | 669 | 1372 | 485 | 1323 |

Floor = 20, max − min > 80, max/min > 2, TEC1HC/tec1Δ > 2 for both chip sets

What is claimed is:

1. A method of inhibiting invasion of a host by a fungus, comprising contacting the fungus with a compound which inhibits expression of a gene expressed in the filamentation MAPK pathway and which enhances said pathway, in sufficient quantity that inhibition of the expression of said gene occurs, thereby inhibiting invasion of the host by the fungus.

2. A method according to claim 1 wherein the gene is TOT10/YELO33W.

3. A method according to claim 1 wherein the host is a plant and the compound is applied to a plant surface in such manner that it contacts the fungus.

4. A method according to claim 1 wherein the fungus is a yeast.

5. A method of inhibiting invasion of a host by a fungus, comprising contacting the fungus with a compound which inhibits activity of a gene product encoded by a gene expressed in the filamentation MAPK pathway, in sufficient quantity that inhibition of the activity of said gene product occurs, thereby inhibiting invasion of the host by the fungus.

6. A method according to claim 5 wherein the gene is TOT10/YELO33W.

7. A method according to claim 5 wherein the host is a plant and the compound is applied to a plant surface in such manner that it contacts the fungus.

8. A method according to claim 5 wherein the fungus is a yeast.

9. A method of identifying an agent which inhibits the filamentation MAPK pathway in a fungus, comprising the steps of:
a) providing an expression vector comprising a nucleic acid molecule of a gene which is expressed in the filamentation MAPK pathway;
b) transforming a suitable host cell with said expression vector under conditions suitable for expression of said gene;
c) contacting said host cell with an agent to be tested; and
d) comparing the expression of said gene in the presence of the agent with the expression of said gene in the absence of said agent,
wherein if the expression of said gene is lower in the presence of the agent than in the absence of the agent, then the agent is an inhibitor of the filamentation MAPK pathway in a fungus.

10. A method according to claim 9, wherein the gene is TOT10/YELO33W.

11. A method according to claim 9, wherein the fungus is yeast.

12. A method according to claim 9, wherein the gene is identified by expression profiling as having repressed expression in the presence of galacturonic acid.

13. A method according to claim 9, wherein the gene is identified by expression profiling as being expressed in haploid fungal cells and not expressed in diploid fungal cells.

14. A method of inhibiting fungal filamentation, comprising contacting the fungus with a compound which inhibits expression of a gene expressed in the filamentation MAPK pathway, in sufficient quantity that inhibition of the expression of said gene occurs, thereby inhibiting filamentation by the fungus.

15. A method of identifying an agent which modulates PGUI gene expression, comprising the steps of:
a) providing an expression vector comprising a nucleic acid molecule encoding PGUI;
b) transforming a suitable host cell with said expression vector under conditions suitable for expression of PGUI;
c) contacting said host cell with an agent to be tested; and
d) comparing the expression of PGUI in the presence of the agent with the expression of PGUI in the absence of said agent,
wherein a difference in the expression of PGUI in the presence of the agent as compared with in the absence of the agent indicates that the agent modulates PGUI expression.

16. A method of reducing the adverse effects of fungal invasion of a host, comprising administering to the host an effective amount of an agent which inhibits PGUI expression in the fungus.

17. A method of inhibiting invasion of a host by a fungus, comprising contacting the fungus with a compound which enhances expression of a gene expressed in the filamentation MAPK pathway and which inhibits said pathway, in sufficient quantity that enhancement of the expression of said gene occurs, thereby inhibiting invasion of the host by the fungus.

18. A method according to claim 1 wherein the fungus is a yeast.
